(19) United States
(12) Patent Application Publication
(10) Pub. No.: US 2012/0094379 A1
(43) Pub. Date: Apr. 19, 2012

(54) HUMAN ENDOMETRIOSIS CELL

(75) Inventors: NANCY M. WANG, Changhua City (TW); Chun-Jung Chen, Taichung City (TW); Hsin-Hung Wu, Changhua City (TW)

(73) Assignee: NATIONAL CHANGHUA UNIVERSITY OF EDUCATION, Changhua City (TW)

(21) Appl. No.: 13/242,256

(22) Filed: Sep. 23, 2011

(30) Foreign Application Priority Data
Oct. 14, 2010 (TW) 099135136

(51) Int. Cl. C12N 5/071

(52) U.S. Cl. 435/366

(57) ABSTRACT

The present invention discloses a human endometriosis cell, EM 257, which has been stored in the food industry research and development institute. Results obtained from a flow cytometry reveal that the human endometriosis cell of the present invention is positive for the mesnchymal stem cell surface antigens, such as CD44, CD73, CD105 and CD146. In different differentiation conditions, the human endometriosis cell of the present invention can be differentiated into osteogenic or adipogenic lineage cells.

### CROSS-REFERENCE TO RELATED APPLICATION

This application claims the benefits of Taiwan Patent Application No. 099135136, filed on Oct. 14, 2010, in the Taiwan Intellectual Property Office, the disclosure of which is incorporated herein in its entirely by reference.

### BACKGROUND OF THE INVENTION

1. Field of the Invention

The present invention generally relates to cells, in particular to a human endometriosis cell.

2. Description of the Related Art

Stem cells are cells with multiple differentiation capability- and self-proliferation capability, and existing in a small number of adult tissues, umbilical cord blood, fetal tissues and embryos. In specific conditions, the stem cells can differentiate into cells with different functions. Since the stem cells have a self-repair ability, scientists conduct extensive researches on the subject of stem cell therapy, and have achieved some effects in the treatment of certain diseases. The stem cells can be mainly divided into embryonic stem cells and adult stem cells. The adult stem cells exist in all human tissues and have the ability of differentiating into specific types of cells to provide the function of repairing human bodies. Among a variety of adult stem cells, there are two main types: hematopoietic stem cells and mesenchymal stem cells.

The mesenchymal stem cells can differentiate into bone and adipose tissues, and subsequent discoveries show that the mesenchymal stem cells can differentiate into cells of liver, cartilage, and muscle tissues. The mesenchymal stem cells are highly plastic stem cells and play a crucial role in the research of the stem cell therapy.

However, the adult stem cell is rare and small in number, and the number of adult stem cells cannot be increased easily by in vitro cell culture. Based on the foregoing reasons, it is an important subject to develop a mesenchymal stem cell that grows stably during the in vitro cell culture.

### SUMMARY OF THE INVENTION

In view of the problems of the prior art, it is a primary objective of the present invention to provide a human endometriosis cell that grows stably during the in vitro cell culture.

To achieve the foregoing objective, the present invention provides a human endometriosis cell , EM 257, which has been stored in the food industry research and development institute, and its storage number is BCRC 960418. The surface marker of the human endometriosis cell of the present invention is analyzed by a flow cytometer, and the human endometriosis cell of the present invention is positive for CD44, CD73, CD105 and CD146, showing that the human endometriosis cell of the present invention is a mesenchymal stem cell.

The human endometriosis cell of the present invention has the ability of differentiation, such that when different bioactive factors are co-cultured with the human endometriosis cell of the present invention, the human endometriosis cell of the present invention can differentiate into an osteogenic cell, an adipogenic lineage cell or a liver cell according to the bioactive factors.

In summation, the human endometriosis cell of the present invention has one or more of the following advantages:

(1) In a continuous in vitro cell culture process without any stimulating bioactive factor, the human endometriosis cell of the present invention can maintain its proliferation for at least one year, so that the human endometriosis cell of the present invention is a very stable cell.

(2) The human endometriosis cell of the present invention can have an in vitro proliferation and a multiple differentiation ability, so that the clinically difficult cases can be overcome by combining the cells of the present invention with tissue engineering techniques, or using the cells of the present invention for a stem cell therapy directly to achieve a good treatment effect.

### BRIEF DESCRIPTION OF THE DRAWINGS

FIG. 1 shows the morphology of in vitro cultured human endometriosis cells in accordance with the present invention;

FIG. 2 shows the expression of CD34 and CD45 on the human endometriosis cells of the present invention;

FIG. 3. shows the expression of CD73, CD105, CD146 and CD44 on the human endometriosis cells of the present invention;

FIG. 4 shows the expression of CD9 and CD13 on the human endometriosis cells of the present invention;

FIG. 5 shows the adipogenic lineage cells differentiated from the Wharton's jelly-derived stem cells, wherein FIG. 5(A) shows the differentiation morphology before the cells are stained by the oil red staining method, and FIG. 5(B) shows the differentiated cells stained by the oil red staining method;

FIG. 6 shows the adipogenic lineage cells differentiated from the human endometriosis cells of the present invention, wherein FIG. 6(A) shows the differentiation morphology before the cells are stained by the oil red staining, and FIG. 6(B) shows the differentiated cells stained by the oil red staining method;

FIG. 7A shows the Wharton's jelly-derived stem cells stained by an alizarin red staining method;

FIG. 7B shows the osteogenic cells differentiated from the Wharton's jelly-derived stem cells and stained by an alizarin red staining method;

FIG. 8A shows the human endometriosis cells of the present invention stained by an alizarin red staining method; and

FIG. 8B shows the osteogenic cells differentiated from the human endometriosis cells of the present invention and stained by an alizarin red staining method.

### DESCRIPTION OF THE PREFERRED EMBODIMENTS

In a first embodiment, the human endometriosis cells of the present invention are isolated from an endometriosis tissue removed by a surgical operation (IRB Approval Certification No.: CCH-070911) and dropped into Dulbecco's modified eagle medium (DMEM). After being shredded and filtered by a 100-μm filter, the tissue is treated with 0.05% of collagenase at 37° C. for 2 hours until the tissue is homogenous.

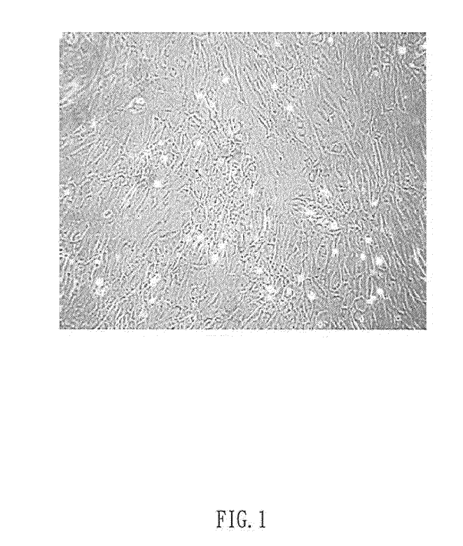

The isolated cells are suspended in the DMEM containing 10% of fetal bovine serum (FBS), 100 units of penicillin and 100 μg of streptomycin. The cells are cultured at 37° C. in a saturated humid environment with 5% of carbon dioxide, and the medium is changed twice a week. With reference to FIG. 1 for the morphology of in vitro cultured human endometriosis cells in accordance with the present invention, when the number of the cells reach an extent of covering 80% area of the Petri dish, the cells are treated and collected with 0.25% of trypsin—EDTA solution and then divided into dishes in a proportion ranging from 1:2 to 1:3. After the cells are grown to a specific quantity in the Petri dish, the analysis and experiment can be conducted.

In a second embodiment, a cell surface marker of the human endometriosis cell of the present invention is given.

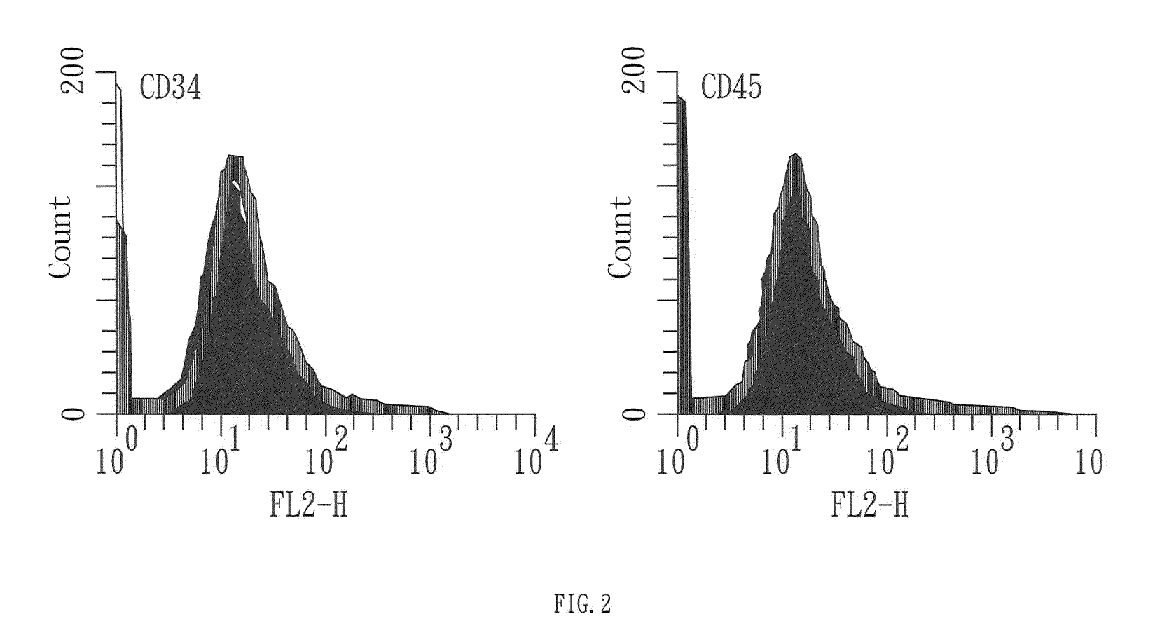

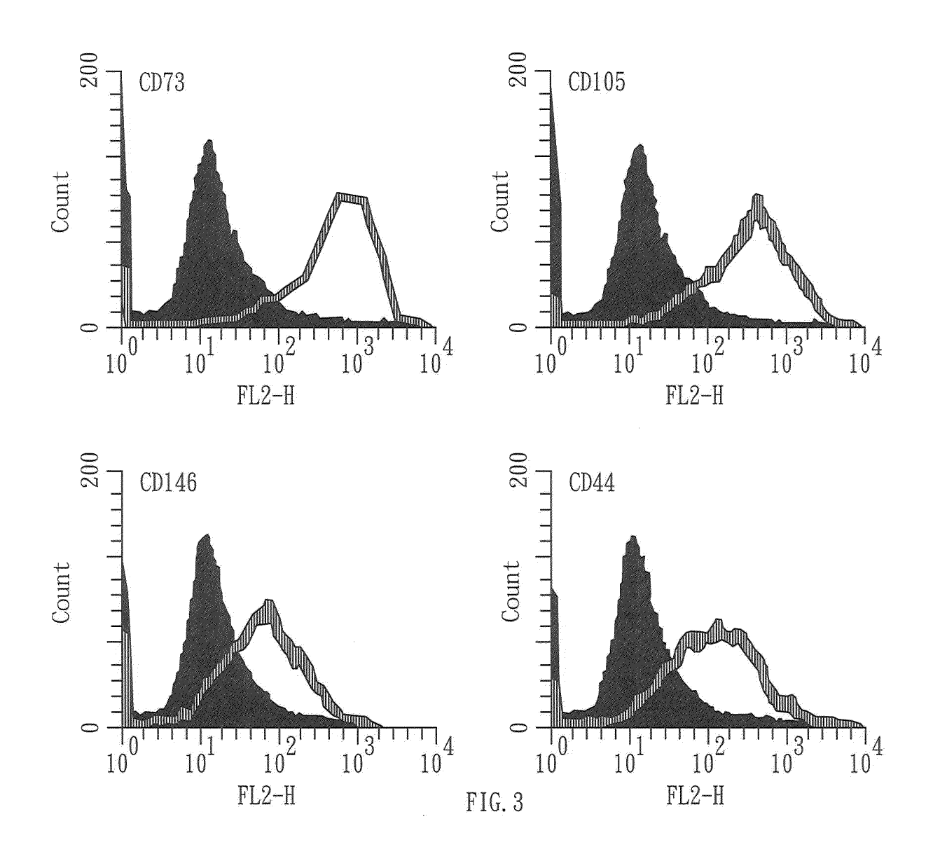

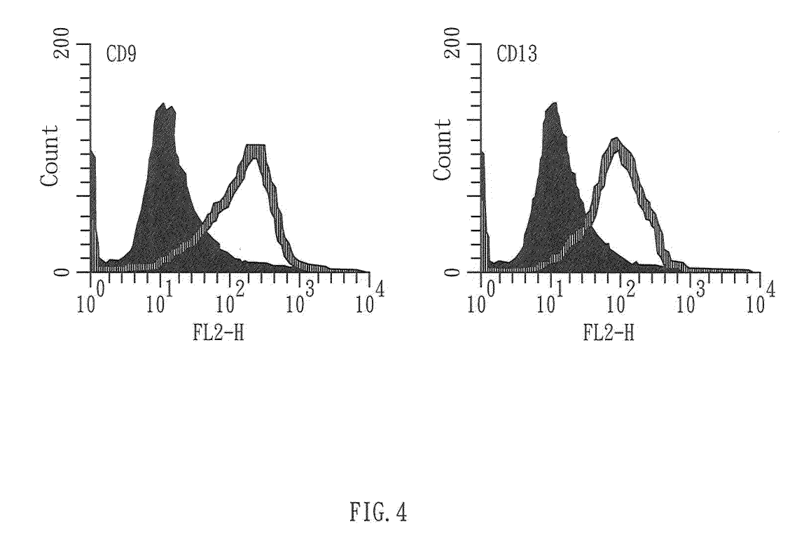

In this embodiment, a flow cytometer is used for executing a flow cytometry analysis. Trypsin is used for processing the 7^{th} subcultured human endometriosis cells, and the following antibodies are used for marking the human endometriosis cells, and the antibodies include (1) antibodies for hematopoietic stem cell surface marker: anti-CD34 and anti-CD45 antibodies; (2) antibodies for mesenchymal stern cell surface marker: anti-CD73, anti-CD105, anti-CD146, and anti-CD44 antibodies; and (3) antibodies for endometrial epithelial cell and stromal cell surface markers: anti-CD9 and anti-CD13 antibodies, and the results are shown in FIGS. 2, 3 and 4.

The results show that the human endometriosis cells of the present invention are negative to the hematopoietic stem cell surface markers CD34 and CD45 and positive to CD44, CD73, CD105 and CD146. The human endometriosis cells of the present invention also express CD9 and CD13, which are cell surface markers of human endometrial epithelial cells and stromal cells respectively.

In summation of the description above, the human endometriosis cell of the present invention is proven to be a mesenchymal stem cell instead of a hematopoietic stem cell and the human endometriosis cell of the present invention is further proven to have the differentiation ability. The following embodiment shows the differentiation of the adipogenic lineage cells and the differentiation of the osteogenic cells. However, the invention is not limited to such differentiations only. If other cells are cultured, another culture medium and its bioactive factors will be adopted.

In a third embodiment, the human endometriosis cells of the present invention obtained from the first embodiment are cultured with the adipogenic medium, which includes 0.5 mM of isobutylmethylxantihine (IBMX), 1 μM of dexamethasone, 10 μM of insulin and 60 μM of indomethacin. An oil red staining method is used to stain neutral lipid vacuoles accumulated in the cells to evaluate the conditions of the adipogenic lineage cells.

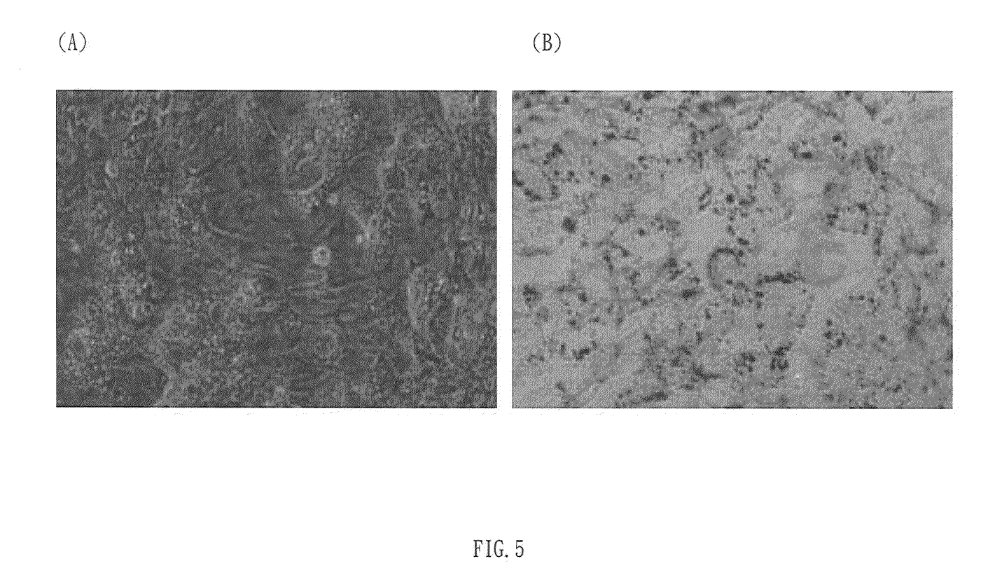

In this embodiment, the Wharton's jelly-derived stem cells are used as a positive control, and the Wharton's jelly-derived stem cell is a gelatin connective tissue separated from the umbilical cord and mainly composed of stromal cells, collagen fibers and glycoproteins. Since the Wharton's jelly-derived stem cell has the feature of the stem cell, therefore it can differentiate into other types of cells. The differentiation results of the Wharton's jelly-derived stem cells are shown in FIGS. 5(A) and 5(B) respectively showing the Wharton's jelly-derived stem cells before and after the oil red staining method is applied for detecting the differentiation of the adipogenic lineage cells, wherein the darker color indicates an accumulation of the adipogenic lineage cells.

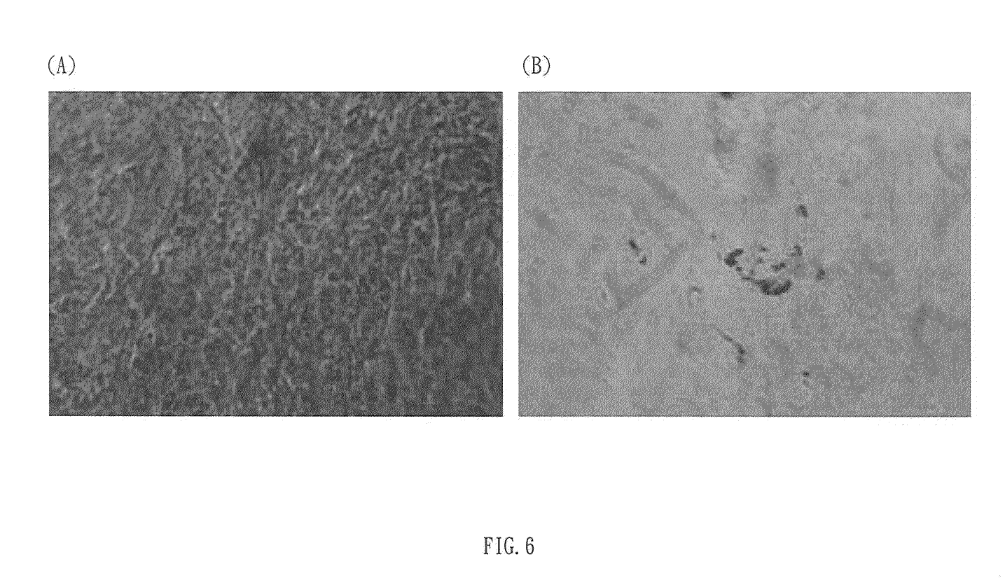

After the human endometriosis cells of the present invention are differentiated, the oil red staining method is also used for observing the differentiation situation of the adipogenic lineage cell, and the results are shown in FIG. 6. FIG. 6(A) shows the differentiation morphology before the oil red staining is applied, and FIG. 6(B) shows the differentiated cells stained by the oil red staining method. A comparison of FIGS. 5 and 6 proves that the human endometriosis cell of the present invention has the ability of differentiating into the adipogenic lineage cell.

In a fourth embodiment, the human endometriosis cell of the present invention obtained from the first embodiment are cultured with osteogenic medium, which contains 0.1 μM of dexamethasone, 10 mM of β-glycerolphosphate and 50 μm of ascorbate (or Vitamin C). An alizarin red staining method is used to detect the calcium accumulation in the cells to evaluate the differentiation of the osteogenic cells.

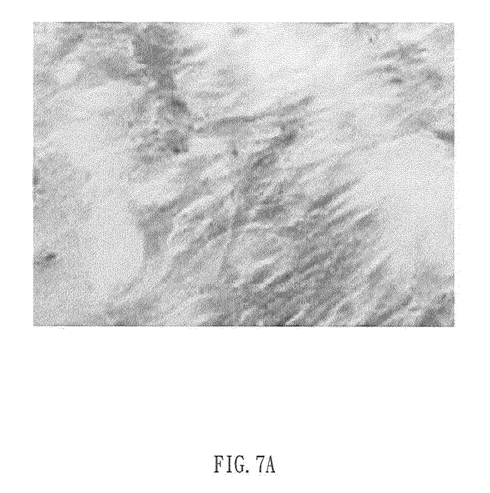

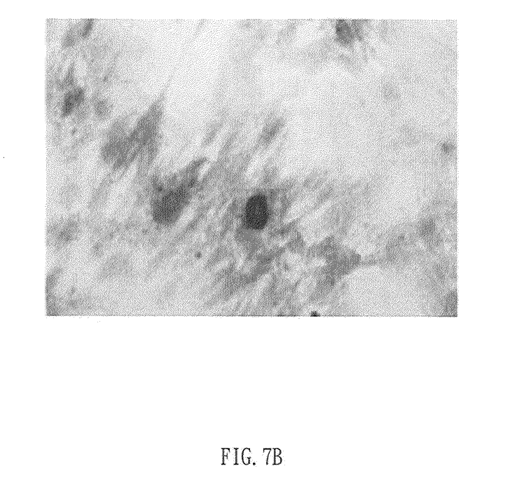

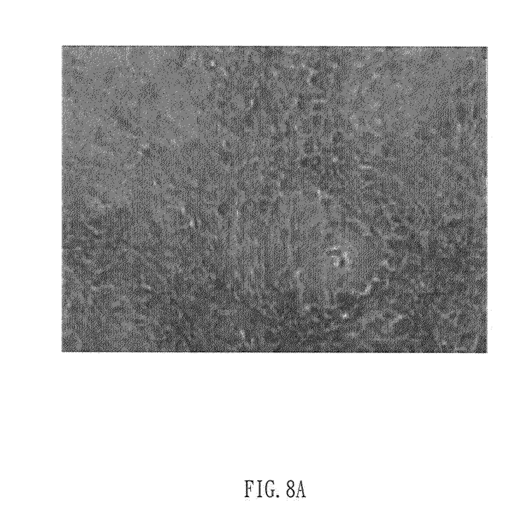

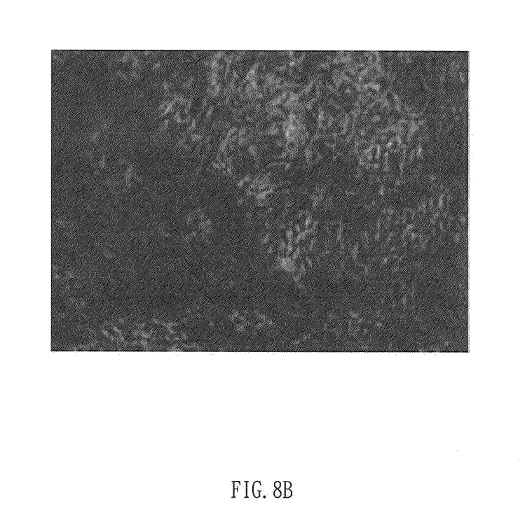

With reference to FIGS. 7A, 7B, 8A and 8B, FIGS. 7A, 7B, 8A and 8B show the alizarin red staining diagrams of the undifferentiated and differentiated Wharton's jelly-derived stem cells (positive control), and the undifferentiated and differentiated human endometriosis cells of the present invention respectively. The results show that after differentiation and stained with alizarin red, a positive reaction on calcium deposition of an orange-red color (at the position of a darker color) of the Wharton's jelly-derived stein cells and the human endometriosis cells of the present invention can be observed, and thus shows that the human endometriosis cell of the present invention has differentiated into the osteogenic cell.

The aforementioned results further show that the human endometriosis cell of present invention is a mesenchymal stem cell, and the human endometriosis cells of the present invention can differentiate into different types of cells such as the osteogenic cells or adipogenic lineage cells when the cells are stimulated by different substances. In addition, the human endometriosis cell of the present invention has applied for a biological storage to the Food Industry Research and Development Institute (Hsinchu, Taiwan) on Aug. 25, 2010, and the storage number is BCRC 960418.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A human endometriosis cell, EM 257, stored in the Food Industry Research and Development Institute, and having a storage number of BCRC 960418.

2. The human endometriosis cell of claim 1, being a mesenchymal stem cell.

3. The human endometriosis cell of claim 1, differentiated to produce an osteogenic cell, an adipogenic lineage cell or a liver cell.

4. The human endometriosis cell of claim 1, having a surface marker of CD44, CD73, CD105 and CD146.
